(19) United States
(12) Patent Application Publication
(10) Pub. No.: US 2019/0183507 A1
(43) Pub. Date: Jun. 20, 2019

(54) TISSUE FASTENING TOOL

(71) Applicant: Spiration, Inc. d/b/a Olympus Respiratory America, Redmond, WA (US)

(72) Inventor: Jean-Martin Baillargeon, Seattle, WA (US)

(21) Appl. No.: 15/844,846

(22) Filed: Dec. 18, 2017

(51) Int. Cl. A61B 17/11

(52) U.S. Cl. CPC A61B 17/1114; A61B 2017/00876

(57) ABSTRACT

A tissue fastening system for fastening a first biological tissue to a second biological tissue. The system includes a tissue fastening tool is made of an elastic wire rod and is wound in a coil shape. The applicator includes a needle for piercing tissue and expelling the tissue fastening tool. The tissue fastening tool includes first and second implants. One of the implants includes magnets and the other is ferromagnetic. At least one of the implants is in an elongate shape when within the needle and in in a circle or coil shape after being expelled from the needle at a first side of the biological tissue.

### BACKGROUND

Surgical bypass (anastomosis) of the gastroenterological (GI), vascular, or urological tracts are typically formed by cutting holes in the tracts at two locations and joining the holes with sutures or staples. The procedure is invasive, and subjects a patient to surgical risks such as bleeding, infection, pain, and adverse reaction to anesthesia. Additionally, an anastomosis created with sutures or staples can be complicated by post-operative leaks and adhesions. Leaks may result in infection or sepsis. Additionally, while traditional anastomosis procedures can be completed with an endoscope, laparoscope, or robot, it can be time consuming to join the holes in the tissues.

As an alternative to sutures or staples, surgeons can use mechanical couplings or magnets to create a compressive anastomosis between tissues. Using surgical techniques, such as endoscopy or laparoscopy, couplings or magnets are placed over the tissues to be joined. Because of the strong compression, the tissue trapped between the couplings or magnets is cut off from its blood supply. Under these conditions, the tissue becomes necrotic and degenerates, and at the same time, new tissue grows around points of compression, i.e., on the edges of the coupling. When the coupling is removed, a healed anastomosis between the two tissues is formed.

Nonetheless, the difficulty of placing the magnets or couplings limits the locations that compressive anastomosis can be used. In most cases, the magnets or couplings have to be delivered as a completed assembly, requiring either an open surgical field or a bulky delivery device. For example, existing magnetic compression devices are limited to structures small enough to be deployed with a delivery conduit e.g., an endoscopic instrument channel or laparoscopic port. When these smaller structures are used, the formed anastomosis is small and suffers from short-term patency, typically lasting several weeks at best.

To overcome the limitations of the mechanical or magnetic coupling devices described above, researchers have developed deployable magnetic anastomosis devices that can be delivered via a delivery conduit. Upon delivery, the devices are intended to self-assemble in useful shapes to form the anastomosis. Such devices are reported in U.S. Pat. No. 8,118,821, incorporated by reference in its entirety. Unfortunately, these designs are not sufficiently robust to be adapted for human use.

### SUMMARY

The present invention relates to a tissue fastening system which fastens a first biological tissue and a second biological tissue. The tissue fastening system includes an applicator device and a tissue fastening tool. The applicator device includes a handle at a proximal end and a deployment needle at a distal end. The tissue fastening tool includes a first implant device and a second implant device. The first implant device includes a ferromagnetic material and the second implant device includes at least one magnet. At least one of the first or second implant devices includes a shape memory material configured to form a circle in a deployed state.

In one aspect of the invention, the second implant device includes a plurality of magnets and a containment structure configured to contain each of the magnets in separate compartments. The containment structure is made at least partially of the shape memory material.

In another aspect of the invention, the containment structure is in a straight or near-straight configuration when located within the deployment needle.

In yet another aspect of the invention, the magnets are oriented within the compartments of the containment structure such that the same magnetic poles for all the magnets are co-located with a first side of the circle when the second implant device is in the deployed state.

In a further aspect of the invention, the shape memory material includes nitinol.

In still other aspects of the invention, the implant device includes a flexible capsule and a ferromagnetic material included within the flexible capsule. The shape memory material is included within the flexible capsule. The ferromagnetic material includes iron particles.

In still further aspects of the invention, the second implant device includes a magnet having a hole.

In still yet other aspects of the invention, the system includes one or more devices configured to connect the first implant device to the second implant device.

In still further aspects of the invention, the deployment needle includes a piercing tip configured to penetrate the first and second biological tissues.

In other aspects of the invention, the applicator device includes a push rod configured to deploy at least one of the first or second implant devices from the deployment needle.

Further features, advantages, and areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way. The components in the figures are not necessarily to scale, emphasis instead being placed upon illustrating the principles of the invention. In the drawings:

FIG. 1 shows an applicator used with an endoscope;

FIG. 2 shows a target region and an endoscope for deploying a device formed in accordance with an embodiment of the present invention;

FIG. 3-1 shows a side view of an exemplary tissue fastening device formed in accordance with the present invention;

FIG. 3-2 shows an end view of the exemplary tissue fastening device of FIG. 3-1;

FIG. 4 shows a side view of a first component of the tissue fastening device of FIG. 3-1 in a straightened configuration;

FIG. 5 shows a view of the first component of the tissue fastening device of FIG. 3-1 in a curved configuration;

FIGS. 6-1 thru 6-10 show action of a component of a tissue fastening device during implantation;

FIG. 7 shows an x-ray view of a magnet ring component in a delivery configuration within a delivery lumen;

FIG. 8 shows a side x-ray view of a magnet ring component in a delivery configuration within a delivery lumen;

FIG. 9 shows a side x-ray view of a needle of the tissue fastening tool deployed within adjacent tissue;

FIG. 10 shows a side x-ray view during deployment of the magnet ring from the deployed needle;

FIG. 11 shows a slice view after the deployment shown in FIG. 10;

FIG. 12 shows a side view of an another tissue fastening tool in place between two body lumen;

FIG. 13 shows a side x-ray view of a first component of the tissue fastening device of FIG. 12 in a straightened configuration; and

FIG. 14 shows an and x-ray view of the first component of FIG. 12 in a curved configuration;

FIG. 15 shows a step of deployment of a first component of a tissue fastening device after deployment within the gallbladder;

FIG. 16 shows a step of deployment of a second port of the tissue fastening device of FIG. 15 within the duodenum;

FIG. 17 shows the magnetic attraction of the tissue fastening device of FIG. 15;

FIG. 18 shows an exemplary components of a tissue fastening device; and

FIG. 19 shows an exemplary components of a tissue fastening device.

### DETAILED DESCRIPTION

A tissue fastening apparatus in the present embodiment is an apparatus which integrally fixes a first biological tissue to a second biological tissue and causes the tissues to communicate with each other. Note that the first and second biological tissues are not limited to different organs. For example, a region of an organ may be referred to as the first biological tissue and a different region of the same organ may be referred to as the second biological tissue, so as to include fixing these two regions within the same organ. In one embodiment, a treatment to fix a common biliary duct as the second biological tissue to a duodenum as the first biological tissue and causes both the organs to communicate with each other will be described as an example.

An exemplary magnetic anastomosis procedure involves placing a magnetic device and a ferromagnetic device adjacent to targeted tissues, thus causing the tissues to come together. The devices are deployed so that that the magnet device will attract the ferromagnetic device and bring the tissues together. The compressed tissues will be allowed to necrose and degrade, providing an opening between the tissues. While the figures and devices of the disclosure are primarily concerned with annular or polygonal structures, it is to be understood that the delivery and construction techniques described herein can be used to make a variety of deployable devices. For example, self-assembling devices can re-assemble into a circle, but could re-assemble into other shapes, such as an ellipse, a square, a hexagon, an octagon, a decagon, or other geometric structure creating a closed loop. The devices may additionally include handles, suture loops, barbs, and protrusions, as needed to achieve the desired performance and to make delivery (and removal) easier.

As described with respect to the figures, a self-assembling magnetic anastomosis device can be placed with a number of techniques, such as endoscopy, laparoscopy, or with a catheter (e.g. not with direct visualization, fluoro, etc.). As described previously, the compression anastomosis process can be allowed to proceed over the ensuing days, resulting in the natural formation of an opening between the tissues. The fused devices can either be allowed to expel naturally or the magnets can be retrieved in a follow-up surgical procedure. Alternatively, if immediate bypass is required, the tissues within the devices can be cut, perforated, pierced or cored. Perforation can be accomplished with a variety of techniques, such as cautery, microscalpel, or coring needle.

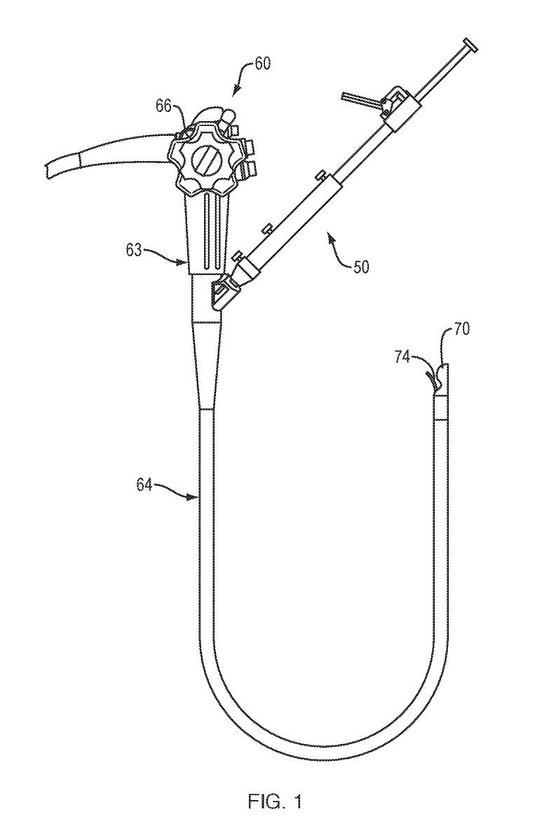

FIG. 1 shows a linear scanning type ultrasonic endoscope 60 (hereinafter simply referred to as an “endoscope”) as an example of an endoscope to be used together with a tissue fastening apparatus 50. The endoscope 60 is provided with a flexible insertion portion 64 that extends from a handle portion 63 to be used outside a body.

The handle portion 63 includes a knob 66 for curving the distal end portion of the insertion portion 64. An ultrasonic device 70 is attached to the distal end of the insertion portion 64.

The distal end of the flexible insertion portion 64 includes a port 74 for delivery of a tissue fastening tool (not shown) of the tissue fastening apparatus 50. The endoscope 60 may be provided with other probe-types of ultrasonic devices. An endoscope with a camera and light may be used. Additionally, an endoscope that is not provided with the ultrasonic device 70 can also be used. In this case, any imaging device used outside the body, such as an X-ray device, a magnetic resonance imaging (MRI) device, or a computerized tomography (CT) device may be used jointly.

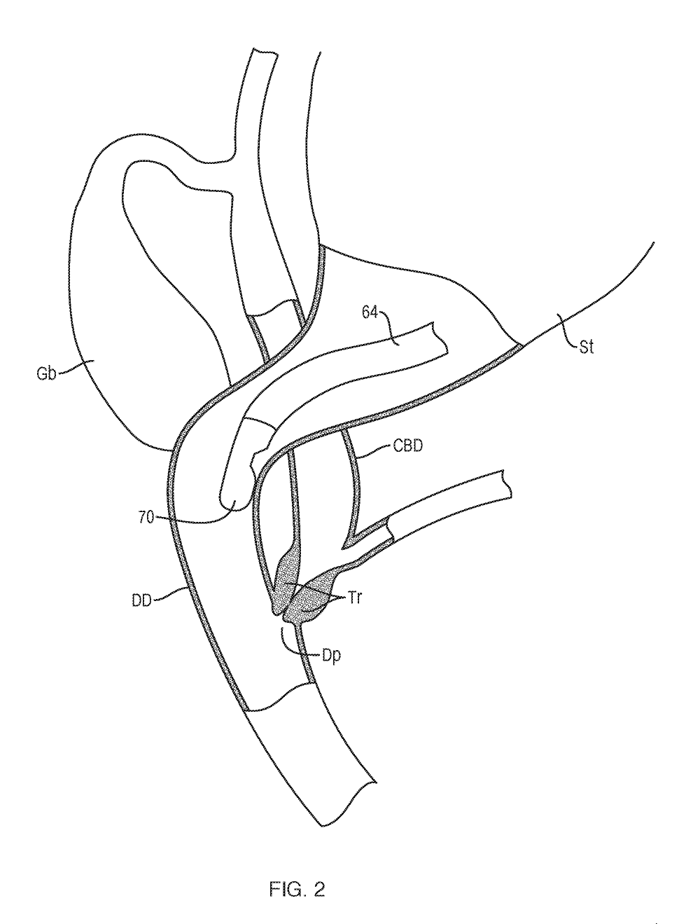

Next, the procedure of indwelling the tissue fastening tool within the body cavity using the tissue fastening apparatus 50 configured as described above, and integrally fixing a duodenum and a common biliary duct to make both to communicate with each other will be described. For example, as shown in FIG. 2, this kind of procedure is performed when a duodenal papilla (Dp) is obstructed, for example by a tumor (Tr), preventing bile drainage, and consequently the bile assimilates in the blood causing jaundice. This procedure enables the direct drainage of bile from the common biliary duct (CBD) to the duodenum (DD).

First, the flexible insertion portion 64 of the endoscope 60 is inserted into the patient's mouth then into the DD. The condition outside the DD is investigated by the ultrasonic device 70 or another imaging device, and an appropriate location for the procedure close to the CBD is targeted proximal from the Dp.

A tissue fastening tool is deployed in stages from the port 74 in order to create a permanent fistula (hole) between the CBD and the DD allowing bile juice from the liver to drain into the DD. This is achieved by the tissue fastening tool compressing the CBD and DD membranes together causing stoppage of blood flow where the tissue is being compressed. The immediate result is interrupted blood flow where the tissue fastening tool is located and over time, membrane fusion around the tissue fastening tool and tissue necrosis where blood flow stopped. The necrotic tissue along with the tissue fastening tool will eventually fall into the duodenum, creating the permanent fistula through which the bile juice will drain.

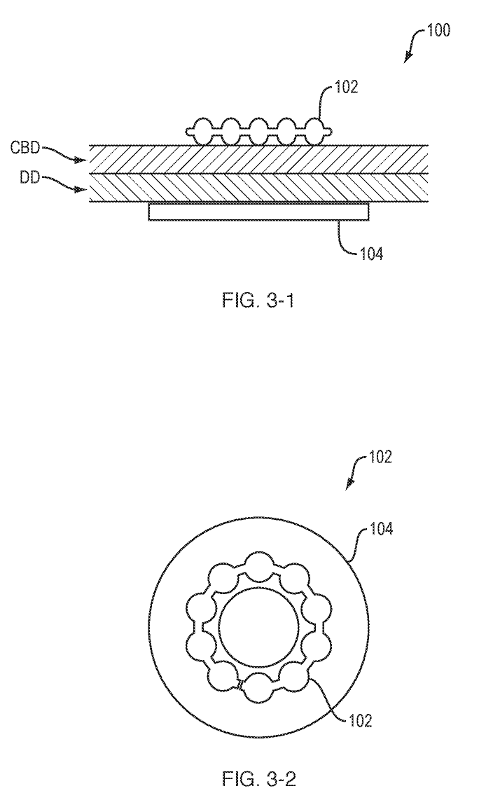

As shown in FIGS. 3-1, 3-2, 4 and 5, a tissue fastening tool 100 includes two distinct components (a magnet ring 102 and a ferromagnetic washer 104) that come together to form an implant. The magnet ring 102 is deployed into the CBD and includes a series of small magnets 106 held together side-by-side using a nitinol frame 108. The geometry of the frame 108 is an interrupted annulus such that in its natural state the magnets form a ring (FIG. 5). The frame 108 would first be created as linear structure then shape set into a “ring” by bringing its two ends together. This allows the frame 108 to be straightened when inserted into a lumen of a deployment needle (not shown) of the tissue fastening apparatus 50. The magnets 106 would have their poles oriented in the same direction to create an attraction side and a repulsion side.

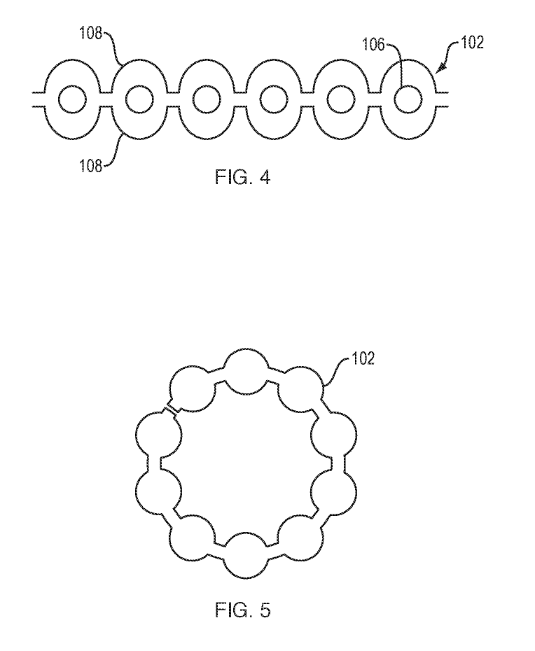

In one embodiment, as shown in FIGS. 4 AND 5, the frame 108 takes the form of a cage. The cage is created in two identical halves then joined together to create pockets for holding cylindrically-shaped magnets. Each half of the cage includes a thin ribbon Nitinol (shape memory) strip material that is shape set to create half-pockets for the magnets. When combining the two ribbon halves together the magnets are inserted into each pocket with all of their negative poles oriented on the same side. The magnets can be attached to the cage in a variety of ways, such as gluing (bonding agent) or laser spot welding them directly to the cage. If not using glue, both cage halves can be held together by spot welding the halves together where the halves contact each other in-between the magnet pockets.

The ferromagnetic washer 104 which is deployed into the DD includes a thin ferrous “washer.” The size of a center hole of the ferromagnetic washer 104 is based on the inner diameter of the magnets 106 when the magnet ring 102 forms a ring. An outer diameter of the ferromagnetic washer 104 is larger than the magnet ring 102. As shown in FIGS. 3-1 and 3-2, the outer diameter of the ferromagnetic washer 104 forms a flange (larger diameter) that prevents the tissue fastening apparatus 100 from falling into the CBD after necrosis, thus ensuring that it will fall into the DD for ejection.

In one embodiment, the washer 104 could be deployed in a variety of ways.

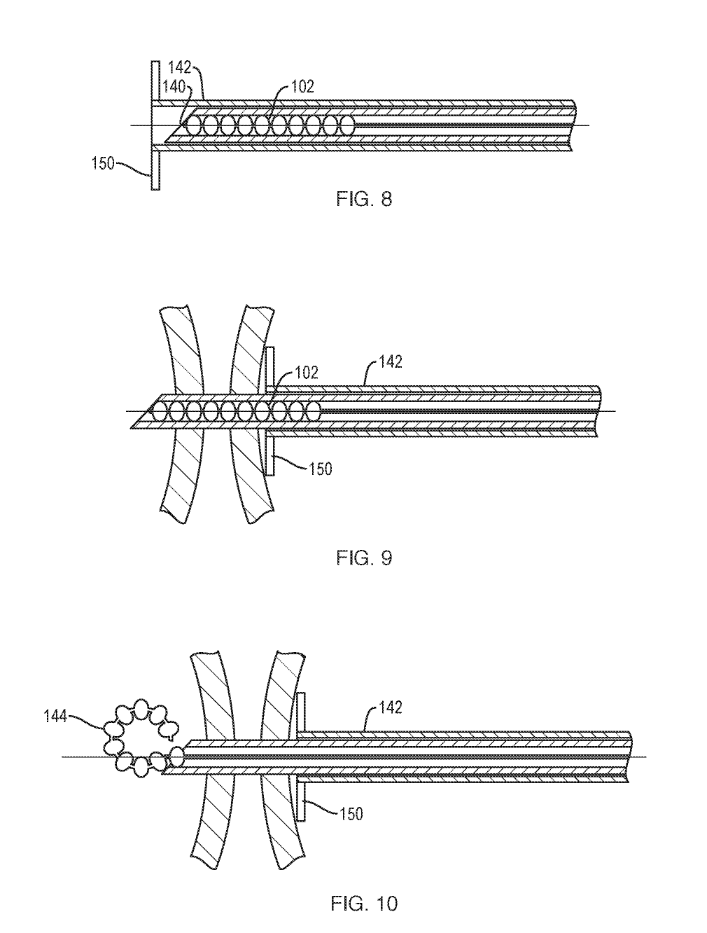

In one example, the washer 104 is pre-loaded onto a deployment sheath (FIGS. 8 through 10).

In one example, the washer 104 is pre-loaded onto a deployment rod (FIG. 17).

In one example, the washer 104 is attached (by suture for example) to the distal end of the endoscope and moved close to the magnet ring after the ring has been deployed, then released.

In one example, after deploying the magnet ring, the endoscope with needle/sheath are completely removed from the body leaving 2 (or more) sutures (holding on to the magnet ring) exposed from the patient's mouth. The exposed sutures ends are then threaded into an inner hole of a washer. The sutures are then fed into the endoscope's distal end of the working channel until they exit at the proximal end (handle end). The washer is sized to that the distal end of the working channel does not pass through the washer. The endoscope is then re-inserted into the patient's GI tract as it was before. The endoscope is advanced to travel to the site where the magnet ring was previously deployed guided by the sutures. Doing so will push and advance the washer to the target site. The distal end of the endoscope can be used to push the washer against the DD tissue to “engage” the magnet ring. After deployment the sutures are cut leaving only a short length still attached to the magnet ring.

After the magnet ring 102 is deployed inside the CBD and positioned flat against the CBD membrane, the ferromagnetic washer 104 is deployed in the DD facing the magnet ring 102. The attraction force between the magnets 106 and the washer 104 then compresses the membranes together and stops the blood from flowing resulting in eventual necrosis.

Deployment of the magnet ring 102 from a non-endobronchial ultrasound scope (EBUS) 110 is generally illustrated in FIGS. 6-1 through 610. The deployment can also occur from a needle within the scope 110. When used with the techniques described herein, the devices allow for the delivery of a larger magnetic structures than would otherwise be possible via a small delivery conduit, such as in a standard endoscope, if the devices were deployed as a completed assembly. Larger magnet structures, in turn, allow for the creation of larger anastomoses that are more robust, and achieve greater surgical success. Because the magnetic devices are radiopaque and echogenic, the devices can be positioned using fluoroscopy, direct visualization (trans-illumination or tissue indentation), and ultrasound, e.g., endoscopic ultrasound. The devices can also be ornamented with radiopaque paint or other markers to help identify the polarity of the devices during placement. In some embodiments, the devices can be positioned by use of sensors located in proximity to the delivery lumen and able to sense the position of a mating device, e.g., using a Reed switch or a Hall-effect sensor.

In one embodiment, a deployment tool (not shown) is used to push the magnet ring 102 out from a deployment lumen, e.g., an endoscope working channel or needle lumen. The deployment tool should be incompressible so as to provide adequate pushability against a device within a delivery lumen, e.g., and endoscope delivery channel. It should also be flexible and lubricious, so that the deployment tool is easily advanced through the endoscope channel after the endoscope has been moved to the site of the desired anastomosis. The deployment tool preferably has a rigid distal tip, either magnetic or non-magnetic. In one embodiment, the deployment tool includes a tip that helps to deploy the magnet ring 102 and helps to guide the distal end of the device to avoid pinching tissue. Furthermore a magnetic tip allows the deployed magnet ring 102 to be easily manipulated with the deployment tool. In other embodiments, a non-magnetic distal tip (stainless steel or similar) allows the deployment rod to manipulate the magnet ring 102 without becoming inadvertently attached to the magnet ring 102.

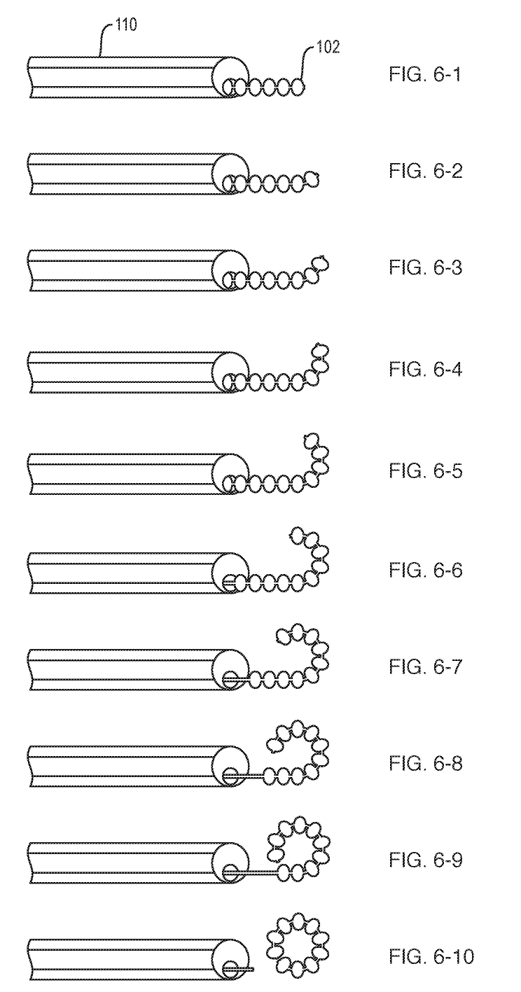

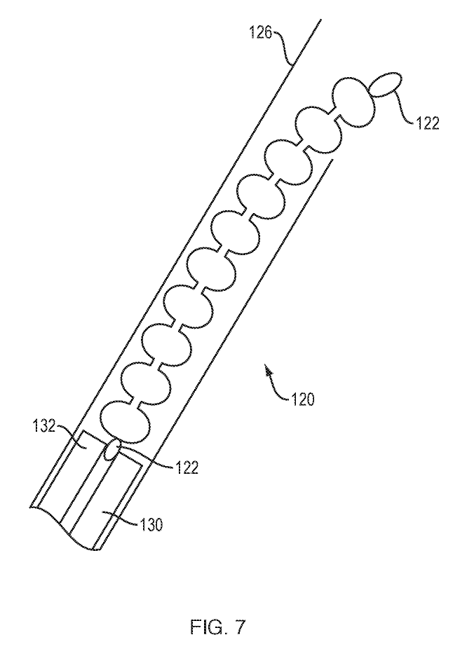

FIG. 7 shows the magnet ring 102 in an open configuration within a delivery needle 140 as the magnet ring 102 is extruded from a distal end 126 of the delivery needle 140. The magnet ring 102 closes on itself as it leaves the delivery needle 140. A stylet 130 slidably received within the delivery needle 140 is used to extrude the magnet ring 102. In one embodiment, a retaining mechanism 132 on the distal end of the delivery needle 120 engages an attachment point 122 at a proximal end of the magnet ring 102. For example, a suture loop passing through attachment point 122 and through a lumen within the push rod 130 to its proximal end can be used to retain and control placement of the magnet ring 102.

FIG. 8 shows the magnet ring 102 loaded into the needle 140 that is slidably received within a sheath 142. The needle 140 and the sheath 142 are inserted into an insertion tube of an endoscope (e.g., the insertion portion 64 of the endoscope 60). After the insertion tube is moved to a target location, the distal end of the needle 140 is forced, by an operator applying pressure to a proximal handle device, out of the insertion tube and the sheath 142. A washer 150 is mounted on a distal end of the sheath 142.

The protruding needle 140 pierces walls of the DD and the CBD (see FIG. 9). Then, as shown in FIG. 10, the operator further operates a deployment tool in order to push the magnet ring 102 out of the needle 140 and into the CBD. As the magnet ring 102 becomes expelled from the needle 140, the magnet ring 140 quickly returns to its originally circular shape due to warming by the surrounding tissue.

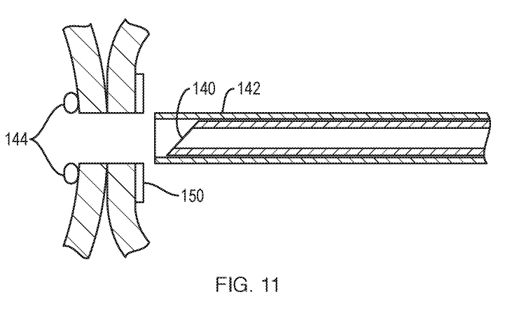

As shown in FIG. 11, after the magnet ring 140 is deployed, the needle 140 is retracted. This causes the magnet ring 140 to magnetically attract the washer 150, thus causing the washer 150 to dislodge from the sheath 142. The magnetic attraction between the washer 150 and the magnet ring 140 compress the walls of the DD and the CBD. A hole may remain between the walls of the DD and the CBD as a result of the penetration of the needle 140. The hole will allow for drainage of fluid into the DD.

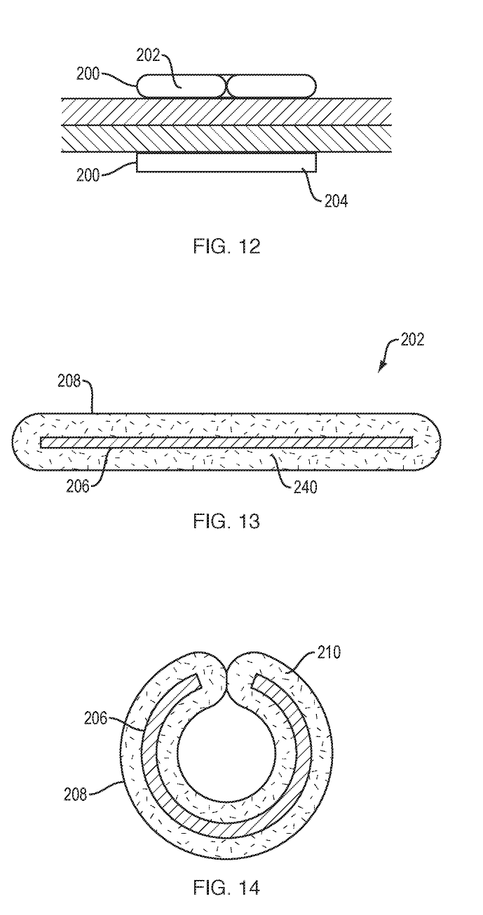

As shown in FIGS. 12-14, an exemplary tissue fastening device 200 includes a sausage-shaped flexible capsule 202 and a washer-shaped magnet 204. The washer-shaped magnet 204 may be a solid washer magnet or may be similar to the magnet ring 102 described above. The sausage-shaped flexible capsule 202 includes a shape memory metal core 206 located within a flexible membrane casing 208. The shape memory metal core 26 may be formed of nitinol shape set to form a closed or nearly closed circle. Also included within the flexible membrane casing 208 are iron oxide particles 210. The flexible membrane casing 208 is made of any biocompatible moldable material that is flexible enough such as silicone, polyurethane (PU), Teflon (PTFE), or Polyvinylchloride (PVC) Polyethersulfone (PES). The iron oxide particles 210 may be in a powder or flake form.

Once the sausage-shaped flexible capsule 202 is deployed from an applicator device (e.g., the delivery needle 140 or comparable devices), the metal core 206 heats up and assumes its shape set form (i.e., circle). The sausage-shaped flexible capsule 202 is deployed within the CBD in a manner similar to the nitinol cage device (the magnet ring 102 in FIG. 4) described above. After the washer-shaped magnet 204 is deployed within the DD, the washer-shaped magnet 204 attracts the sausage-shaped flexible capsule 202 causing a wall of the CBD and the DD to come under pressure.

Once deployed, the magnet ring 102 or the sausage-shaped flexible capsule 202 (implant devices) can be precisely delivered to the appropriate anatomical location using the deployment tool. In some embodiments, this will be accomplished with a deployment tool providing additional degrees of freedom, specifically axial displacement and rotation, about the scope axis. As described above, the distal tip of the deployment tool may be magnetic, ferromagnetic or non-ferromagnetic, to provide control of the implant devices before, during, and after deployment. In one embodiment, the deployment tool tip is shaped with a miter that matches the proximal end of the implant device. To release the implant device from the deployment tool, the deployment tool is simply withdrawn, or the endoscope advanced, until the delivery tool pulls into the endoscope's working channel and the endoscope pushes on, and releases, the implant device. In some procedures the deployment tool may be redeployed after it has been decoupled from the implant device, for example to manipulate a deployed implant device into the proper location. Alternatively, the deployment tool can be used to locate a magnetic structure that has been place on the other side of a tissue plane, but is not visible through the endoscope.

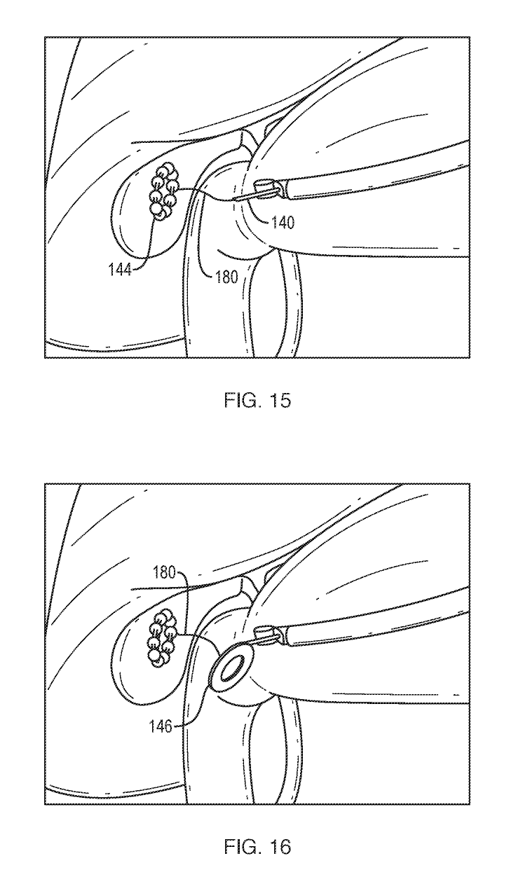

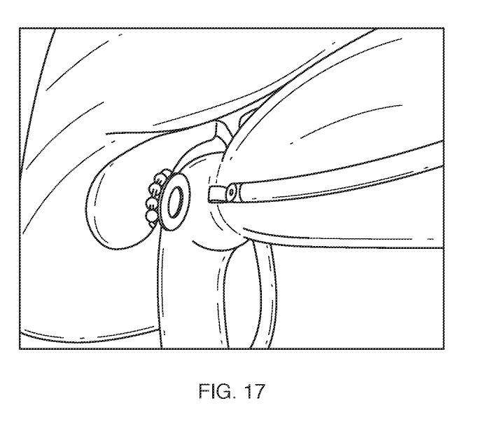

As shown in FIG. 15, a deployed first implant device 144 is shown deployed within the gallbladder. A suture 180 is connected at the distal end of the first deployed implant device 144. The proximal end of the suture 180 passes to the proximal end of the deployment needle 140 or to a second implant device 146. Once the needle 140 is retracted through the wall of the DD, the suture 180 remains connected at the first implant device 144. Then, as shown in FIG. 16, the second implant device 146 is deployed and, as shown in FIG. 17, magnetically bonded to the first implant device 144. Manipulation of the suture 180 may help guide the bonding between the implant devices 144, 146.

Magnet fixation can be visually guided using EUS-guided T-tag delivery through the tissue walls. Such a T-tag procedure is well known to persons skilled in the art of therapeutic endoscopy. Fluoroscopy is one tool for confirming magnetic attraction between the implant devices 144, 146. When the implant devices 144, 146 are left in place for a period of time, the compressive forces on the tissue between the two magnets causes the tissue to necrose, leaving an opening.

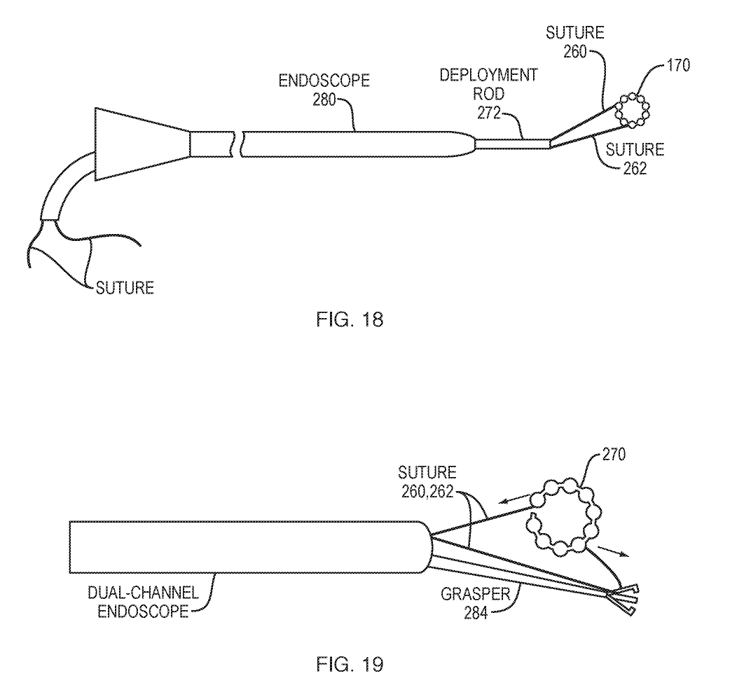

As shown in FIG. 18, two high tensile suture members 260, 262 are attached and integrated into an implant device 170. The two tensile suture members 260, 262 are attached to the implant device 170 at points approximately 180 degrees apart when in the deployed configuration. Each of the suture members 260, 262 then runs through a deployment rod 272, through the deployment rod length and are accessible at the proximal end of an endoscope 280 that receives the rod 272. The tensile suture members 260, 262 can be attached to a handle or tension wheel on the proximal end, to simplify placement of the implant device 270. For example, pulling on both sutures will allow a surgeon to center the implant device 270. The implant device 270 can also be moved left/right and rotated by manipulating the tensile suture members 260, 262. The suture members 260, 262 provide the ability to pull back and assist in capture of the implant devices between two different and separated anatomical structures.

As appreciated by those of skill in the art, device placement is especially important in certain applications, such as creation of a bile duct or other anastomosis. In this situation, it is important that the implant devices encompass the puncture site, to ensure the area is sealed and not leaking. To ensure that the device is delivered approximately on center of the puncture site, the tensile suture members can be used to center the implant devices to assure that the puncture site is sealed. Once the two implant devices are deployed and magnetically bonded at the desired target location, the sutures can be cut.

Additionally, should the implant devices need to be removed, one or more graspers 284 can be introduced (endoscopically or otherwise), allowing the two tensile suture members 260, 262 to be used to provide an opening force as shown in FIG. 19. The lateral force created by pulling on one or more attachment points causes a reorientation of the implant device, thereby causing the ring to open up. For example, one of the sutures can be pulled through the deployment device while the other is pulled through the grasper 284, thus providing the needed opposing force. In this instance, the deployment device can be pulled back into the endoscopic channel, thereby pulling the implant device 270 back into the deployment channel.

### Embodiments

A. A tissue fastening tool for applying pressure between a first biological tissue and a second biological tissue, the tool comprising: a first implant device; and a second implant device, wherein the first implant device comprises a ferromagnetic material and the second implant device comprises at least one magnet, wherein at least one of the first or second implant devices comprises a shape memory material configured to form a circle in a deployed state.

B. The tissue fastening tool according to A, wherein the second implant device comprises: a plurality of magnets; and a containment structure configured to contain each of the magnets in separate compartments, wherein the containment structure is made at least partially of the shape memory material.

C. The tissue fastening tool according to any of A or B, wherein the containment structure is in a straight or near-straight configuration when in a pre-deployment state.

D. The tissue fastening tool according to any of A-C, wherein the magnets are oriented within the compartments of the containment structure such that the same magnetic poles for all the magnets are co-located with a first side of the circle when the second implant device is in the deployed state.

E. The tissue fastening tool according to any of A-D, wherein the shape memory material includes nitinol.

F. The tissue fastening tool according to any of A-E, wherein the first implant device further comprises: a flexible capsule; and a ferromagnetic material included within the flexible capsule, wherein the shape memory material is included within the flexible capsule.

G. The tissue fastening tool according to F, wherein the ferromagnetic material comprises iron particles.

H. The tissue fastening tool according to any of F or G, wherein the second implant device comprises a magnet having a hole.

I. The tissue fastening tool according to any of A-H, further comprising one or more devices configured to connect the first implant device to the second implant device.

J. The tissue fastening system for applying pressure between a first biological tissue and a second biological tissue, the system comprising: an applicator device comprising: a handle at a proximal end; and a deployment needle at a distal end; and a tissue fastening tool comprising: a first implant device; and a second implant device, wherein the first implant device comprises a ferromagnetic material and the second implant device comprises at least one magnet, wherein at least one of the first or second implant devices comprises a shape memory material configured to form a circle in a deployed state.

K. The tissue fastening system according J, wherein the second implant device comprises: a plurality of magnets; and a containment structure configured to contain each of the magnets in separate compartments, wherein the containment structure is made at least partially of the shape memory material.

L. The tissue fastening system according to any of J or K, wherein the containment structure is in a straight or near-straight configuration when located within the deployment needle.

M. The tissue fastening system according to any of K or L, wherein the magnets are oriented within the compartments of the containment structure such that the same magnetic poles for all the magnets are co-located with a first side of the circle when the second implant device is in the deployed state.

N. The tissue fastening system according to any of J-M, wherein the shape memory material includes nitinol.

O. The tissue fastening system according to any of J-N, wherein the implant device comprises: a flexible capsule; and a ferromagnetic material included within the flexible capsule, wherein the shape memory material is included within the flexible capsule.

P. The tissue fastening system according to O, wherein the ferromagnetic material comprises iron particles.

Q. The tissue fastening system according to any of O or P, wherein the second implant device comprises a magnet having a hole.

R. The tissue fastening system according to any of J-Q, further comprising one or more devices configured to connect the first implant device to the second implant device.

S. The tissue fastening system according any of O-R, wherein the deployment needle comprises a piercing tip configured to penetrate the first and second biological tissues.

T. The tissue fastening system according to any of O-S, wherein the applicator device further comprises a push rod configured to deploy at least one of the first or second implant devices from the deployment needle.

The description of the invention is merely exemplary in nature and variations that do not depart from the gist of the invention are intended to be within the scope of the invention. Such variations are not to be regarded as a departure from the spirit and scope of the invention.

Although the preferable embodiments of the present invention have been described hitherto, the present invention is not limited to these embodiments. Additions, omissions, substitutions, and other modifications can be made without departing from the spirit or scope of the present invention.

In addition, the invention is not limited by the above description and is limited by only the scope of appended claims.

What is claimed is:

1. A tissue fastening tool for applying pressure between a first biological tissue and a second biological tissue, the tool comprising:
a first implant device; and
a second implant device,
wherein the first implant device comprises a ferromagnetic material and the second implant device comprises at least one magnet,
wherein at least one of the first or second implant devices comprises a shape memory material configured to form a circle in a deployed state.

2. The tissue fastening tool according to claim 1, wherein the second implant device comprises:
a plurality of magnets; and
a containment structure configured to contain each of the magnets in separate compartments,
wherein the containment structure is made at least partially of the shape memory material.

3. The tissue fastening tool according to claim 2, wherein the containment structure is in a straight or near-straight configuration when in a pre-deployment state.

4. The tissue fastening tool according to claim 3, wherein the magnets are oriented within the compartments of the containment structure such that the same magnetic poles for all the magnets are co-located with a first side of the circle when the second implant device is in the deployed state.

5. The tissue fastening tool according to claim 1, wherein the shape memory material includes nitinol.

6. The tissue fastening tool according to claim 1, wherein the first implant device further comprises:
a flexible capsule; and
a ferromagnetic material included within the flexible capsule,
wherein the shape memory material is included within the flexible capsule.

7. The tissue fastening tool according to claim 6, wherein the ferromagnetic material comprises iron particles.

8. The tissue fastening tool according to claim 6, wherein the second implant device comprises a magnet having a hole.

9. The tissue fastening tool according to claim 1, further comprising one or more devices configured to connect the first implant device to the second implant device.

10. A tissue fastening system for applying pressure between a first biological tissue and a second biological tissue, the system comprising:
an applicator device comprising:
a handle at a proximal end; and
a deployment needle at a distal end; and
tissue fastening tool comprising:
a first implant device; and
a second implant device,
wherein the first implant device comprises a ferromagnetic material and the second implant device comprises at least one magnet,
wherein at least one of the first or second implant devices comprises a shape memory material configured to form a circle in a deployed state.

11. The tissue fastening system according to claim 10, wherein the second implant device comprises:
a plurality of magnets; and
a containment structure configured to contain each of the magnets in separate compartments,
wherein the containment structure is made at least partially of the shape memory material.

12. The tissue fastening system according to claim 11, wherein the containment structure is in a straight or near-straight configuration when located within the deployment needle.

13. The tissue fastening system according to claim 12, wherein the magnets are oriented within the compartments of the containment structure such that the same magnetic poles for all the magnets are co-located with a first side of the circle when the second implant device is in the deployed state.

14. The tissue fastening system according to claim 10, wherein the shape memory material includes nitinol.

15. The tissue fastening system according to claim 10, wherein the implant device comprises:
a flexible capsule; and
a ferromagnetic material included within the flexible capsule,
wherein the shape memory material is included within the flexible capsule.

16. The tissue fastening system according to claim 15, wherein the ferromagnetic material comprises iron particles.

17. The tissue fastening system according to claim 15, wherein the second implant device comprises a magnet having a hole.

18. The tissue fastening system according to claim 10, further comprising one or more devices configured to connect the first implant device to the second implant device.

19. The tissue fastening system according to claim 10, wherein the deployment needle comprises a piercing tip configured to penetrate the first and second biological tissues.

20. The tissue fastening system according to claim 19, wherein the applicator device further comprises a push rod configured to deploy at least one of the first or second implant devices from the deployment needle.
